(19) United States
(12) Patent Application Publication
(10) Pub. No.: US 2024/0366760 A1
(43) Pub. Date: Nov. 7, 2024

(54) ACTIVATION OF SURVIVIN-SPECIFIC IMMUNE RESPONSES USING DENDRITIC CELL DERIVED EXOSOMES

(71) Applicant: Regen Biopharma, Inc., La Mesa, CA (US)

(72) Inventors: Thomas ICHIM, San Diego, CA (US); David KOOS, La Mesa, CA (US)

(73) Assignee: Regen Biopharma, Inc., La Mesa, CA (US)

(21) Appl. No.: 18/415,206

(22) Filed: Jan. 17, 2024

Related U.S. Application Data
(60) Provisional application No. 63/439,526, filed on Jan. 17, 2023.

(51) Int. Cl. A61K 39/00; A61P 37/04; C07K 14/47; C12N 5/0784; C12N 15/113

(52) U.S. Cl. CPC A61K 39/4615; A61P 37/04; C07K 14/4747; C12N 5/0639; C12N 15/1136; A61K 2039/5158; A61K 2039/585; C12N 2310/14; C12N 2501/22; C12N 2501/2304; C12N 2506/45

(57) ABSTRACT

Disclosed are means, methods and compositions of matter useful for stimulation of immunity to tumor antigens through isolation of dendritic cell exosomes from dendritic cells that have been pulsed with said tumor antigens and/or pulsed dendritic cells that have been gene silenced/gene edited for immune suppressive genes. In one embodiment said tumor antigen is survivin and said immune suppressive genes include interleukin-10, interleukin-35, TGF-beta and interleukin-13. In some embodiments dendritic cells are further transfected with immune stimulatory genes including interleukin-2, interleukin−12, interleukin-15 and interleukin-18.

### CROSS-REFERENCE TO RELATED APPLICATIONS

This application claims priority to U.S. Provisional Application No. 63/439,526, filed Jan. 17, 2023, and titled “Activation of Survivin-Specific Immune Responses using Dendritic Cell Derived Exosomes Alone and/or from siRNA/Gene Edited Dendritic Cells”, which is hereby incorporated by reference in its entirety.

### SEQUENCE LISTING

The instant application contains a Sequence Listing which has been submitted electronically in XML file format and is hereby incorporated by reference in its entirety. Said XML copy, created on Jul. 25, 2024, is named Reg_Surv_NP1_SL.xml and is 6,306 bytes in size.

### FIELD OF THE INVENTION

The teachings herein relate to compositions and methods of treating cancer utilizing exosomes from dendritic cells, including genetically modified dendritic cells.

### BACKGROUND OF THE INVENTION

New methods and compositions are needed to treat cancer in mammals.

### SEQUENCE LISTING

SEQ ID NO. 1: GCAACAACGCCAUCUAUGA

SEQ ID NO. 2: UCAUAGAUGGCGUUGUUGC

SEQ ID NO. 3: QIWQLYLKNYRIATFKNWP

SEQ ID NO. 4: ATFKNWPF

SEQ ID NO. 5: AKFVAAWTLKAAA

SEQ ID NO. 6: YLKNYRIQIWQLATFKNWP

### SUMMARY OF THE INVENTION

Preferred embodiments are directed to methods of stimulating an immune response to survivin comprising: a) obtaining a progenitor cell population; b) transfecting said progenitor cell population with survivin and/or survivin epitopes; c) optionally transfecting/gene editing one or more immune stimulatory genes; d) optionally gene silencing/gene editing one or more genes associated with immune suppression; e) differentiating said progenitor cells into dendritic cells; and f) collecting exosomes and/or microvesicles from said dendritic cells and utilizing them as a cancer immunotherapy.

Preferred methods include embodiments, wherein said progenitor cell is a pluripotent stem cell.

Preferred methods include embodiments wherein said pluripotent stem cell is an inducible pluripotent stem cell.

Preferred methods include embodiments wherein said inducible pluripotent stem cell is generated by exposure to one or more de-differentiating agents.

Preferred methods include embodiments wherein said dedifferentiating agent is cytoplasm from a pluripotent stem cell.

Preferred methods include embodiments, wherein said cytoplasm from said pluripotent stem cell is administrated intracellularly through microinjection.

Preferred methods include embodiments, wherein said cytoplasm from said pluripotent stem cell is administrated intracellularly through temporary permeabilization of the cellular membrane of recipient cell.

Preferred methods include embodiments, wherein said temporary permeabilization is achieved through electroporation.

Preferred methods include embodiments, wherein said temporary permeabilization is achieved through osmotic shock.

Preferred methods include embodiments, wherein said temporary permeabilization is achieved through administration of membrane permeabilizing agents.

Preferred methods include embodiments wherein said membrane permeabilizing agent is streptolysin 0.

Preferred methods include embodiments wherein said membrane permeabilizing agent is a defensin.

Preferred methods include embodiments wherein said defensin is Neutrophil defensin 1.

Preferred methods include embodiments wherein said defensin is Defensin, alpha 1.

Preferred methods include embodiments wherein said defensin is Neutrophil defensin 3.

Preferred methods include embodiments wherein said defensin is Neutrophil defensin 4.

Preferred methods include embodiments wherein said defensin is Defensin-5.

Preferred methods include embodiments wherein said defensin is Defensin-6.

Preferred methods include embodiments wherein said defensin is Beta-defensin 1.

Preferred methods include embodiments wherein said defensin is Beta-defensin 2.

Preferred methods include embodiments wherein said defensin is Beta-defensin 3.

Preferred methods include embodiments wherein said defensin is Beta-defensin 103.

Preferred methods include embodiments wherein said defensin is Beta-defensin 106A.

Preferred methods include embodiments wherein said defensin is Beta-defensin 106B.

Preferred methods include embodiments wherein said defensin is Beta-defensin 107.

Preferred methods include embodiments wherein said defensin is Beta-defensin 110.

Preferred methods include embodiments wherein said defensin is Beta-defensin 138.

Preferred methods include embodiments wherein said defensin is Beta-defensin 107.

Preferred methods include embodiments wherein said membrane permeabilizing agent is polymyxin B.

Preferred methods include embodiments wherein said membrane permeabilizing agent is a polymyxin E.

Preferred methods include embodiments wherein said membrane permeabilizing agent is compound 48/80.

Preferred methods include embodiments wherein said membrane permeabilizing agent is Cecropin A.

Preferred methods include embodiments wherein said membrane permeabilizing agent is Cecropin B.

Preferred methods include embodiments wherein said membrane permeabilizing agent is CECD

Preferred methods include embodiments wherein said membrane permeabilizing agent is Papiliocin.

Preferred methods include embodiments wherein said membrane permeabilizing agent is Cecropin P1.

Preferred methods include embodiments wherein said membrane permeabilizing agent is Cecropin P1.

Preferred methods include embodiments wherein said cell undergoing dedifferentiation to generate said pluripotent stem cell is a fibroblast.

Preferred methods include embodiments wherein said fibroblast is derived from placenta.

Preferred methods include embodiments wherein said fibroblast is derived from placental chorion.

Preferred methods include embodiments wherein said fibroblast expresses CD105.

Preferred methods include embodiments wherein said fibroblast expresses CD90.

Preferred methods include embodiments wherein said fibroblast expresses CD69.

Preferred methods include embodiments wherein said fibroblast expresses interleukin-3 receptor.

Preferred methods include embodiments wherein said fibroblast expresses c-met.

41. Preferred methods include embodiments wherein said cell undergoing de-differentiation is a monocyte.

42. Preferred methods include embodiments wherein said monocyte is proliferating.

43. Preferred methods include embodiments wherein said monocyte expresses ability to differentiate into a CD5 B cell.

44. Preferred methods include embodiments wherein said monocyte expresses CD5.

Preferred methods include embodiments wherein said monocyte expresses c-kit.

Preferred methods include embodiments wherein said monocyte expresses c-kit and interleukin-3 receptor.

Preferred methods include embodiments wherein said monocyte expresses CD5.

Preferred methods include embodiments wherein said monocyte expresses HLA-G.

Preferred methods include embodiments wherein said monocyte expresses ILT3.

Preferred methods include embodiments wherein said monocyte expresses ILT4.

Preferred methods include embodiments wherein said monocyte expresses IL-10 receptor.

Preferred methods include embodiments wherein said monocyte expresses IL-4 receptor.

Preferred methods include embodiments wherein said monocyte expresses IL-13 receptor.

Preferred methods include embodiments wherein said monocyte expresses IL-20 receptor.

Preferred methods include embodiments wherein said monocyte expresses IL-25 receptor.

Preferred methods include embodiments wherein said monocyte expresses IL-35 receptor.

Preferred methods include embodiments wherein said monocyte expresses arginase.

Preferred methods include embodiments wherein said monocyte expresses indolamine 2,3 dioxygenase.

Preferred methods include embodiments wherein said monocyte expresses FGF-1 receptor.

Preferred methods include embodiments wherein said monocyte expresses FGF-2 receptor.

Preferred methods include embodiments wherein said monocyte expresses FGF-5 receptor.

Preferred methods include embodiments wherein said monocyte expresses leukemia inhibitory factor receptor.

Preferred methods include embodiments wherein said monocyte expresses hepatocyte growth factor receptor.

Preferred methods include embodiments wherein said cell undergoing de-differentiation is a mesenchymal stem cell.

Preferred methods include embodiments wherein said mesenchymal stem cell is derived from a group of tissue selected from a group comprising of: a) umbilical cord; b) cord blood; c) placenta; d) peripheral blood; e) mobilized peripheral blood; f) menstrual blood; g) bone marrow, and h) fallopian tube.

Preferred methods include embodiments wherein said mesenchymal stem cells express CD105 and/or CD73 and/or CD90.

Preferred methods include embodiments wherein said mesenchymal stem cell lack expression of CD14, and/or CD45 and/or CD34.

Preferred methods include embodiments wherein said mesenchymal stem cells possess ability to differentiate into cartilage.

Preferred methods include embodiments wherein said mesenchymal stem cells possess ability to differentiate into bone.

Preferred methods include embodiments wherein said mesenchymal stem cells possess ability to differentiate into fat.

Preferred methods include embodiments wherein said mesenchymal stem cells secrete interleukin-10 subsequent to contact with an activator of toll like receptor 4.

Preferred methods include embodiments wherein said mesenchymal stem cells secrete interleukin-1 receptor antagonist subsequent to contact with an activator of toll like receptor 4.

Preferred methods include embodiments wherein said mesenchymal stem cells secrete soluble TNF-alpha receptor p55 subsequent to contact with an activator of toll like receptor 4.

Preferred methods include embodiments wherein said mesenchymal stem cells secrete TNF-alpha receptor p75 subsequent to contact with an activator of toll like receptor 4.

Preferred methods include embodiments wherein said mesenchymal stem cells secrete BlyS subsequent to contact with an activator of toll like receptor 4.

Preferred methods include embodiments wherein said mesenchymal stem cells secrete TRAIL subsequent to contact with an activator of toll like receptor 4.

Preferred methods include embodiments wherein said mesenchymal stem cells secrete soluble Fas ligand subsequent to contact with an activator of toll like receptor 4.

Preferred methods include embodiments wherein said mesenchymal stem cells secrete BlyS subsequent to contact with an activator of toll like receptor 4.

Preferred methods include embodiments wherein said progenitor cell is modified to express interleukin-12.

Preferred methods include embodiments wherein said progenitor cell is modified to express interleukin-15.

Preferred methods include embodiments wherein said progenitor cell is modified to express interleukin-18.

Preferred methods include embodiments wherein said progenitor cell is modified to express interleukin-7.

Preferred methods include embodiments wherein said progenitor cell is modified to express interleukin-15 homodimers.

Preferred methods include embodiments wherein said progenitor cell is modified to express interleukin-21.

Preferred methods include embodiments wherein said progenitor cell is modified to express interleukin-23.

Preferred methods include embodiments wherein said progenitor cell is modified to express interleukin-27.

Preferred methods include embodiments wherein said progenitor cell is modified to express interleukin-33.

Preferred methods include embodiments wherein said progenitor cell is modified to express TNF-alpha.

Preferred methods include embodiments wherein said progenitor cell is modified to express TRAIL.

Preferred methods include embodiments wherein said progenitor cell is modified to express TNF-beta.

Preferred methods include embodiments wherein said progenitor cell is modified to express interferon alpha.

Preferred methods include embodiments wherein said progenitor cell is modified to express interferon gamma.

Preferred methods include embodiments wherein said progenitor cell is modified to express complement C3.

Preferred methods include embodiments wherein said progenitor cell is modified to express complement C5.

Preferred methods include embodiments wherein said progenitor cell is modified to express membrane attack complex components.

Preferred methods include embodiments wherein said progenitor cell is modified to express MIP-1 alpha.

Preferred methods include embodiments wherein said progenitor cell is modified to express MIP-1 beta.

Preferred methods include embodiments wherein said progenitor cell is modified to express CXCL12.

Preferred methods include embodiments wherein said progenitor cell is modified to express RANK ligand.

Preferred methods include embodiments wherein said progenitor cell is modified to express NKG2D activators.

Preferred methods include embodiments wherein said NKG2D activator is hsp96.

Preferred methods include embodiments wherein said NKG2D activator is MICA.

Preferred methods include embodiments wherein said NKG2D activator is MICB.

Preferred methods include embodiments wherein said NKG2D activator is H60.

Preferred methods include embodiments wherein said NKG2D activator is Rae-1.

Preferred methods include embodiments wherein said NKG2D activator is MICA.

Preferred methods include embodiments wherein said progenitor cell is modified to express a mono-specific, bispecific or poly-specific protein capable of binding and activating CD3.

Preferred methods include embodiments wherein said progenitor cell is modified to express a mono-specific, bispecific or poly-specific protein capable of binding and activating CD56.

Preferred methods include embodiments wherein said progenitor cell is modified to express a mono-specific, bispecific or poly-specific protein capable of binding and activating CD16.

Preferred methods include embodiments wherein said progenitor cell is modified to express a mono-specific, bispecific or poly-specific protein capable of binding and activating CD31.

Preferred methods include embodiments wherein said progenitor cell is modified to express a mono-specific, bispecific or poly-specific protein capable of binding and activating CD5.

Preferred methods include embodiments wherein said progenitor cell is modified to express a mono-specific, bispecific or poly-specific protein capable of binding and activating CD28.

Preferred methods include embodiments wherein said progenitor cell is modified to express a mono-specific, bispecific or poly-specific protein capable of binding and activating CD4.

Preferred methods include embodiments wherein said progenitor cell is modified to express a mono-specific, bispecific or poly-specific protein capable of binding and activating CD8.

Preferred methods include embodiments wherein said progenitor cell is modified to express a mono-specific, bispecific or poly-specific protein capable of binding and activating interleukin-2 receptor.

Preferred methods include embodiments wherein said progenitor cell is modified to express a mono-specific, bispecific or poly-specific protein capable of binding and activating interleukin-7 receptor.

Preferred methods include embodiments wherein said progenitor cell is modified to express a mono-specific, bispecific or poly-specific protein capable of binding and activating interleukin-12 receptor.

Preferred methods include embodiments wherein said progenitor cell is modified to express a mono-specific, bispecific or poly-specific protein capable of binding and activating interleukin-15 receptor.

Preferred methods include embodiments wherein said progenitor cell is modified to express a mono-specific, bispecific or poly-specific protein capable of binding and activating interleukin-17 receptor.

Preferred methods include embodiments wherein said progenitor cell is modified to express a mono-specific, bispecific or poly-specific protein capable of binding and activating interleukin-18 receptor.

Preferred methods include embodiments wherein said progenitor cell is modified to express a mono-specific, bispecific or poly-specific protein capable of binding and activating interleukin-22 receptor.

Preferred methods include embodiments wherein said progenitor cell is modified to express a mono-specific, bispecific or poly-specific protein capable of binding and activating NKG2D.

Preferred methods include embodiments wherein said progenitor cell is modified to express a mono-specific, bispecific or poly-specific protein capable of facilitating attraction between the cell itself and a monocyte.

Preferred methods include embodiments wherein said progenitor cell is modified to express a mono-specific, bispecific or poly-specific protein capable of facilitating attraction between the cell itself and a T cell.

Preferred methods include embodiments wherein said progenitor cell is modified to express a mono-specific, bispecific or poly-specific protein capable of facilitating attraction between the cell itself and a NK cell.

Preferred methods include embodiments wherein said progenitor cell is modified to express a mono-specific, bispecific or poly-specific protein capable of facilitating attraction between the cell itself and a gamma delta T cell.

Preferred methods include embodiments wherein said progenitor cell is modified to express a mono-specific, bispecific or poly-specific protein capable of facilitating attraction between the cell itself and an NKT cell.

Preferred methods include embodiments wherein said progenitor cell is modified to express a mono-specific, bispecific or poly-specific protein capable of facilitating attraction between the cell itself and a neutrophil.

Preferred methods include embodiments wherein said progenitor cell is modified to express a mono-specific, bispecific or poly-specific protein capable of facilitating attraction between the cell itself and a type 1 neutrophil.

Preferred methods include embodiments wherein said progenitor cell is modified to express a mono-specific, bispecific or poly-specific protein capable of facilitating attraction between the cell itself and a mast cell.

Preferred methods include embodiments wherein said progenitor cell is modified to express a mono-specific, bispecific or poly-specific protein capable of facilitating attraction between the cell itself and an innate lymphoid cell.

Preferred methods include embodiments wherein said progenitor cell is modified to express a mono-specific, bispecific or poly-specific protein capable of facilitating attraction between the cell itself and a Th1 cell.

Preferred methods include embodiments wherein said Th1 cell is capable of producing more interferon gamma than interleukin-4.

Preferred methods include embodiments wherein said Th1 cell is expresses GATA-3.

Preferred methods include embodiments wherein said Th1 cell is capable of inducing production of type 1 monocytes when cultured with monocyte progenitors.

Preferred methods include embodiments wherein said progenitor cell is modified to express a mono-specific, bispecific or poly-specific protein capable of facilitating attraction between the cell itself and a Th17 cell.

Preferred methods include embodiments wherein said progenitor cell is modified to express a mono-specific, bispecific or poly-specific protein capable of facilitating attraction between the cell itself and a Th9 cell.

Preferred methods include embodiments wherein said survivin is administrated as the whole coding section of the survivin gene.

Preferred methods include embodiments wherein a portion of the survivin gene is transfected.

Preferred methods include embodiments wherein said survivin gene is fused with one or more genes or gene fragments to increase immunogenicity.

Preferred methods include embodiments wherein said survivin gene is fused to a heat shock protein.

Preferred methods include embodiments wherein said heat shock protein is gp96.

Preferred methods include embodiments wherein said survivin gene is fused to CpG motifs capable of activating toll like receptor 9.

A composition useful for stimulating an immune response to cancer comprising exosomes produced by dendritic cells expressing survivin or parts of the survivin gene, wherein said dendritic cells are derived from a cell type that was dedifferentiated into immature cells which are subsequently used to generate a dendritic cell population, wherein during said immature phase various immunomodulatory modifications have been made to said immature cells, furthermore wherein said modified immature cells are expanded to generate a master cell bank and subsequently said immature cells are differentiated to dendritic cells and said dendritic cells are utilized as a source of exosomes.

Preferred compositions include embodiments wherein said dendritic cells are generated from pluripotent stem cells.

Preferred compositions include embodiments wherein said pluripotent stem cells are transfected with a construct encoding the survivin gene together with one or more immunogenic molecules.

Preferred compositions include embodiments wherein said immunogenic molecule is a toll like receptor agonist.

Preferred compositions include embodiments wherein said toll like receptor agonist is a DNA sequence encoding a double stranded RNA.

Preferred compositions include embodiments wherein said toll like receptor agonist is DNA encoding the flagellin protein.

Preferred compositions include embodiments wherein said toll like receptor agonist is DNA encoding HMGB-1.

Preferred compositions include embodiments wherein said toll like receptor agonist is DNA encoding gp-96.

Preferred compositions include embodiments wherein said cell type which was dedifferentiated possesses ability to proliferate without differentiation for at least 50 doublings.

Preferred compositions include embodiments wherein said dedifferentiated cell is utilized as a progenitor for generation of dendritic cells.

Preferred compositions include embodiments wherein said dedifferentiated cells are exposed to dendritic cell differentiating factors subsequent to transfection and/or pulsing with survivin gene and/or protein.

Preferred compositions include embodiments wherein said dendritic cell

differentiating factor is conditioned media of monocytes exposed to a toll like receptor 4 agonist.

Preferred compositions include embodiments wherein said dendritic cell differentiating factor is a mixture of IL-7, IL-4, and GM-CSF.

Preferred compositions include embodiments wherein said dendritic cell differentiating factor is a mixture of IL-7, IL-4, and GM-CSF together with conditioned media from monocytes exposed to a toll like receptor 4 agonist.

Preferred compositions include embodiments wherein said toll like receptor 4 agonist is lipopolysaccharide.

Preferred compositions include embodiments wherein said toll like receptor 4 agonist is hyaluronic acid.

Preferred compositions include embodiments wherein said toll like receptor 4 agonist is calreticulin.

Preferred compositions include embodiments wherein said exosomes express annexin-V.

Preferred compositions include embodiments wherein said exosomes express CD8.

Preferred compositions include embodiments wherein said immature cell is obtained by culture with an agent associated with embryonic microenvironment.

Preferred compositions include embodiments wherein said agent is leukemia inhibitory factor.

Preferred compositions include embodiments wherein said leukemia inhibitory factor is administered to cells that have been synchronized in cell cycle.

Preferred compositions include embodiments wherein said synchronization of cells in cell cycle is achieved by treatment with a mitotic inhibitor.

Preferred compositions include embodiments wherein said mitotic inhibitor is mitomycin C.

Preferred compositions include embodiments wherein synchronization is achieved by serum deprivation.

Preferred compositions include embodiments wherein said leukemia inhibitory factor is administered at a concentration of 1 pg/ml to 100 ng/ml.

Preferred compositions include embodiments wherein said leukemia inhibitory factor is administered at a concentration of 10 pg/ml to 10 ng/ml.

Preferred compositions include embodiments wherein said leukemia inhibitory factor is administered at a concentration of 100 pg/ml to 5 ng/ml.

Preferred compositions include embodiments wherein said leukemia inhibitory factor is administered together with a histone deacetylase inhibitor.

Preferred compositions include embodiments wherein said histone deacetylase inhibitor is valproic acid.

Preferred compositions include embodiments wherein said valproic acid is administered at a concentration of 1 pg/ml to 1 mg/ml.

Preferred compositions include embodiments wherein said valproic acid is administered at a concentration of 100 pg/ml to 100 ng/ml.

Preferred compositions include embodiments wherein said valproic acid is administered at a concentration of 1 ng/ml to 100 ng/ml.

Preferred compositions include embodiments wherein said histone deacetylase inhibitor is vorinostat.

Preferred compositions include embodiments wherein said histone deacetylase inhibitor is belinostat.

Preferred compositions include embodiments wherein said histone deacetylase inhibitor is LAQ824.

Preferred compositions include embodiments wherein said histone deacetylase inhibitor is trichostatin A.

Preferred compositions include embodiments wherein said histone deacetylase inhibitor is Panobinostat.

Preferred compositions include embodiments wherein said histone deacetylase inhibitor is entinostat.

Preferred compositions include embodiments wherein said histone deacetylase inhibitor is CI994.

Preferred compositions include embodiments wherein said histone deacetylase inhibitor is mocetinostat.

Preferred compositions include embodiments wherein said histone deacetylase inhibitor is trapoxin B.

Preferred compositions include embodiments wherein said histone deacetylase inhibitor is phenylbutyrate.

Preferred compositions include embodiments wherein a DNA methyltransferase inhibitor is added to said combination leukemia inhibitory factor and said DNA histone deacetylase inhibitor.

Preferred compositions include embodiments wherein said DNA methyltransferase inhibitory is a nucleic acid derivative.

Preferred compositions include embodiments wherein said DNA methyltransferase inhibitory is decitabine.

Preferred compositions include embodiments wherein said DNA methyltransferase inhibitory is 5-azacytabine.

Preferred compositions include embodiments wherein said reprogramming is induced through suppression of the enzyme GSK-3.

Preferred compositions include embodiments wherein said suppression of said enzyme GSK-3 is accomplished by treatment with lithium.

Preferred compositions include embodiments wherein said lithium is administered at a concentration of 1 pg/ml to 1 mg/ml.

Preferred compositions include embodiments wherein said lithium is administered at a concentration of 10 pg/ml to 100 ng/ml.

Preferred compositions include embodiments wherein said lithium is administered at a concentration of 100 pg/ml to 10 ng/ml.

Preferred compositions include embodiments wherein said reprogramming is induced through induction of expression of the gene OCT3/4.

Preferred compositions include embodiments wherein said reprogramming is induced through induction of expression of the gene SOX2.

Preferred compositions include embodiments wherein said reprogramming is induced through induction of expression of the gene KLF4.

Preferred compositions include embodiments wherein said reprogramming is induced through induction of expression of the gene L-MYC.

Preferred compositions include embodiments wherein said reprogramming is induced through induction of expression of the gene LIN28.

Preferred compositions include embodiments wherein said reprogramming is induced through induction of expression of the gene BCL-xL.

Preferred compositions include embodiments wherein said reprogramming is induced through induction of expression of the gene BCL-xL.

Preferred compositions include embodiments wherein said reprogramming is induced through inhibition of p53 expression.

Preferred compositions include embodiments wherein p53 expression is suppression of p53 activity.

Preferred compositions include embodiments wherein said suppression of p53 activity is mediated through administration of a small molecule inhibitor of p53.

Preferred compositions include embodiments wherein said suppression of p53 activity is mediated through administration of decoy oligonucleotides.

Preferred compositions include embodiments wherein said suppression of p53 activity is mediated through administration of decoy peptides.

Preferred compositions include embodiments wherein said suppression of p53 expression is achieved through induction of RNA interference targeting p53.

Preferred compositions include embodiments wherein said induction of RNA interference targeting p53 is induced through administration of short interfering RNA.

Preferred compositions include embodiments wherein said induction of RNA interference targeting p53 is induced through administration of short hairpin RNA.

Preferred compositions include embodiments wherein said suppression of p53 expression is achieved through administration of antisense oligonucleotides targeting p53.

Preferred compositions include embodiments wherein said antisense oligonucleotides induce cleavage of nucleic acids through activation of RNAse H.

Preferred compositions include embodiments wherein said suppression of p53 expression is achieved through administration of ribozymes targeting p53.

Preferred compositions include embodiments wherein said suppression of p53 expression is achieved through gene editing.

Preferred compositions include embodiments wherein said reprogramming is induced by culture of said cells in a liquid media containing ascorbic acid.

Preferred compositions include embodiments wherein said reprogramming is induced by culture of said cells in a liquid media containing transferrin.

Preferred compositions include embodiments wherein said reprogramming is induced by culture of said cells in a liquid media containing sodium bicarbonate.

Preferred compositions include embodiments wherein said reprogramming is induced by culture of said cells in a liquid media containing insulin.

Preferred compositions include embodiments wherein said reprogramming is induced by culture of said cells in a liquid media containing sodium selenite.

Preferred compositions include embodiments wherein said reprogramming is induced by culture of said cells in a hypoxic environment.

Preferred compositions include embodiments wherein said hypoxic environment comprises of oxygen levels low enough to induce activation of hypoxia inducible factor (HIF)-1.

Preferred compositions include embodiments wherein said hypoxic environment is culture of said cells in an environment less than 21% oxygen.

Preferred compositions include embodiments wherein said hypoxic environment is culture of said cells in an environment less than 15% oxygen.

Preferred compositions include embodiments wherein said hypoxic environment is culture of said cells in an environment less than 10% oxygen.

Preferred compositions include embodiments wherein said hypoxic environment is culture of said cells in an environment containing approximately 5% oxygen.

Preferred compositions include embodiments wherein said reprogramming is induced by culture of cells in a liquid media containing a MAP Kinase inhibitor.

Preferred compositions include embodiments wherein said MAP kinase inhibitor is PD0325901/.

Preferred compositions include embodiments wherein said reprogramming is induced by culture of cells in a liquid media containing SB431542.

Preferred compositions include embodiments wherein said reprogramming is induced by culture of cells in a liquid media containing CHIR99021.

Preferred compositions include embodiments wherein said reprogramming is induced by culture of cells in a liquid media containing Y-27632.

Preferred compositions include embodiments wherein said reprogramming is induced by culture of cells in a liquid media containing Y-thiazovivin.

Preferred compositions include embodiments wherein said reprogramming is induced by culture of cells in a liquid media containing FGF-1.

Preferred compositions include embodiments wherein said reprogramming is induced by culture of cells in a liquid media containing FGF-2.

Preferred compositions include embodiments wherein said reprogramming is induced by culture of cells in a liquid media containing FGF-5.

Preferred compositions include embodiments wherein said reprogramming is induced by culture of cells in a liquid media containing sodium borate.

Preferred compositions include embodiments wherein said reprogramming is induced by culture of cells in a liquid media containing erythropoietin (EPO).

Preferred compositions include embodiments wherein said reprogramming is induced by culture of cells in a liquid media containing interleukin-3.

Preferred compositions include embodiments wherein said reprogramming is induced by culture of cells in a liquid media containing interleukin-6.

Preferred compositions include embodiments wherein said reprogramming is induced by culture of cells in a liquid media containing interleukin-8.

Preferred compositions include embodiments wherein said reprogramming is induced by culture of cells in a liquid media containing interleukin-10.

Preferred compositions include embodiments wherein said reprogramming is induced by culture of cells in a liquid media containing interleukin-18.

Preferred compositions include embodiments wherein said reprogramming is induced by culture of cells in a liquid media containing interleukin-20.

Preferred compositions include embodiments wherein said reprogramming is induced by culture of cells in a liquid media containing interleukin-25.

Preferred compositions include embodiments wherein said reprogramming is induced by culture of cells in a liquid media containing insulin-like growth factor-1 (IGF-1).

Preferred compositions include embodiments wherein said reprogramming is induced by culture of cells in a liquid media containing dexamethasone.

Preferred compositions include embodiments wherein said reprogramming is induced by culture of cells in a liquid media containing holo-transferrin.

Preferred compositions include embodiments wherein said reprogramming is induced by culture of cells in a liquid media containing amino acids selected from a group comprising of Glycine, L-histidine, L-isoleucine, L-methionine, L-phenylalanine, L-proline, L-hydroxyproline, L-serine, L-threonine, L-tryptophan, and L-tyrosine, L-valine.

Preferred compositions include embodiments wherein said reprogramming is induced by culture of cells in a liquid media containing vitamins and/or antioxidants selected from a group comprising of thiamine, reduced glutathione, ascorbic acid and 2-PO.sub.4.

Preferred compositions include embodiments wherein said reprogramming is induced by culture of cells in a liquid media containing trace elements selected from a group comprising of: Ag.sup.+, Al.sup.3+, Ba.sup.2+, Cd.sup.2+, Co.sup.2+, Cr.sup.3+, Ge.sup.4+, Se.sup.4+, Br.sup.−, I.sup.−, F.sup.−, Mn.sup.2+, Si.sup.4+, V.sup.5+, MO.sup.6+, Ni.sup.2+, Rb.sup.+, Sn.sup.2+, and Zr.sup.4+.

Preferred compositions include embodiments wherein said reprogramming is induced by culture of cells in a liquid media containing cytoplasm of an undifferentiated cell.

Preferred compositions include embodiments wherein said cell being reprogrammed has its membrane temporarily permeabilized.

The composition of 248, wherein said temporary permeabilization allows for entry of cytoplasm of undifferentiated cell into cytoplasm of said cell to be reprogrammed.

Preferred compositions include embodiments wherein said undifferentiated cell is syngeneic with the cell whose reprogramming is desired.

Preferred compositions include embodiments wherein said undifferentiated cell is allogeneic with the cell whose reprogramming is desired.

Preferred compositions include embodiments wherein said undifferentiated cell is xenogeneic with the cell whose reprogramming is desired.

Preferred compositions include embodiments wherein said permeabilization is mediated by electroporation.

Preferred compositions include embodiments wherein said permeabilization is mediated by Streptolysin O treatment.

Preferred compositions include embodiments wherein said permeabilization is mediated by transient treatment with complement membrane attack complex.

Preferred compositions include embodiments wherein said permeabilization is mediated by transient treatment with perforin.

Preferred compositions include embodiments wherein said permeabilization is mediated by transient treatment with granzyme.

Preferred compositions include embodiments wherein said undifferentiated cell is an oocyte.

Preferred compositions include embodiments wherein said oocyte is programmed to be at G0/G1 of cell cycle.

Preferred compositions include embodiments wherein said programming to be at G0/G1 of cell cycle is accomplished by exposure to mitomycin C.

Preferred compositions include embodiments wherein said programming to be at GO/G1 of cell cycle is accomplished by exposure to serum starvation.

Preferred compositions include embodiments wherein said undifferentiated cell is an inducible pluripotent stem cell.

Preferred compositions include embodiments wherein said undifferentiated cell is a parthenogenic derived stem cell.

Preferred compositions include embodiments wherein said undifferentiated cell is an embryonic stem cell.

Preferred compositions include embodiments wherein said undifferentiated cell is a somatic cell nuclear transfer derived stem cell.

Preferred compositions include embodiments wherein said undifferentiated cell is a cytoplasmically reprogrammed stem cell.

Preferred compositions include embodiments wherein said undifferentiated cell is a cell obtained by fusion of an adult cell with a pluripotent stem cell.

Preferred compositions include embodiments wherein said fusion is accomplished by the use of polyethylene glycol.

Preferred compositions include embodiments wherein said fusion is accomplished by the use of electrically mediated fusion.

### BRIEF DESCRIPTION OF DRAWINGS

FIG. 1 is a bar graph showing the results of tumor growth in mice based on the following administrating exosomes from the following sources: (a) control DC, (b) TGF-beta silenced DC, (c) Control DC and survivin, and (d) TGF-beta silenced DC and survivin.

### DETAILED DESCRIPTION OF THE INVENTION

The invention teaches the use of exosomes derived from dendritic cells expressing survivin epitopes as an immune stimulation means to generate survivin-specific T cell responses. In one embodiment the invention provides the use of pluripotent stem cells transfected with survivin and/or survivin altered peptides which are modified to possess increased immunogenicity wherein such pluripotent cells are differentiated into myeloid and/or dendritic cells which are utilized as a source of exosomes for use in immunotherapy.

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

Chemical Modification: As used herein, “chemical modification” refers to the process wherein a chemical or biochemical is used to induce genomic changes in the donor cell, or nucleus thereof, that allow the donor cell, or nucleus thereof, to be responsive during maturation and receptive to the host cell cytoplasm.

Committed: As used herein, “committed” refers to cells which are considered to be permanently committed to a specific function. Committed cells are also referred to as “terminally differentiated cells.”

Cytoplast Extract Modification: As used herein, “cytoplast extract modification” refers to the process wherein a cellular extract consisting of the cytoplasmic contents of a cell are used to induce genomic changes in the donor cell, or nucleus thereof, that allow the donor cell, or nucleus thereof, to be responsive during maturation and receptive to the host cell cytoplasm.

Dedifferentiation: As used herein, “dedifferentiation” refers to loss of specialization in form or function. In cells, dedifferentiation leads to an a less committed cell.

Differentiation: As used herein, “differentiation” refers to the adaptation of cells for a particular form or function. In cells, differentiation leads to a more committed cell.

Donor Cell: As used herein, “donor cell” refers to any diploid (2N) cell derived from a pre-embryonic, embryonic, fetal, or post-natal multi-cellular organism or a primordial sex cell which contributes its nuclear genetic material to the hybrid stem cell. The donor cell is not limited to those cells that are terminally differentiated or cells in the process of differentiation. For the purposes of this invention, donor cell refers to both the entire cell or the nucleus alone.

Donor Cell Preparation: As used herein, “donor cell preparation” refers to the process wherein the donor cell, or nucleus thereof, is prepared to undergo maturation or prepared to be receptive to a host cell cytoplasm and/or responsive within a post-natal environment.

Germ Cell: As used herein, “germ cell” refers to a reproductive cell such as a spermatocyte or an oocyte, or a cell that will develop into a reproductive cell.

Host Cell: As used herein, “host cell” refers to any multipotent stem cell derived from a pre-embryonic, embryonic, fetal, or post-natal multicellular organism that contributes the cytoplasm to a hybrid stem cell.

Host Cell Preparation: As used herein, “host cell preparation” refers to the process wherein the host cell is enucleated.

Hybrid Stem Cell: As used herein, “hybrid stem cell” refers to any cell that is multipotent and is derived from an enucleated host cell and a donor cell, or nucleus thereof, of a multicellular organism. Hybrid stem cells are further disclosed in co-pending U.S. patent application Ser. No. 10/864,788.

Karyoplast Extract Modification: As used herein, “karyoplast extract modification” refers to the process wherein a cellular extract consisting of the nuclear contents of a cell, lacking the DNA, are used to induce genomic changes in the donor cell, or nucleus thereof, that allow the donor cell, or nucleus thereof, to be responsive during maturation or receptive to the host cell cytoplasm.

Maturation: As used herein, “maturation” refers to a process of coordinated steps either forward or backward in the differentiation pathway and can refer to both differentiation or de-differentiation. As used herein, maturation is synonymous with the terms develop or development when applied to the process described herein.

Modified Germ Cell: As used herein, “modified germ cell” refers to a cell comprised of a host enucleated ovum and a donor nucleus from a spermatogonia, oogonia or a primordial sex cell. The host enucleated ovum and donor nucleus can be from the same or different species. A modified germ cell can also be called a “hybrid germ cell.”

Multipotent: As used herein, “multipotent” refers to cells that can give rise to several other cell types, but those cell types are limited in number. An example of a multipotent cells is hematopoietic cells-blood stem cells that can develop into several types of blood cells but cannot develop into brain cells.

Multipotent Adult Progenitor Cells: As used herein, “multipotent adult progenitor cells” refers to multipotent cells isolated from the bone marrow which have the potential to differentiate into mesenchymal, endothelial and endodermal lineage cells.

Pre-embryo: As used herein, “pre-embryo” refers to a fertilized egg in the early stage of development prior to cell division. During the pre-embryonic stage the initial stages of cleavage are occurring.

Pre-embryonic Stem Cell: See “Embryonic Stem Cell” above.

Post-natal Stem Cell: As used herein, “post-natal stem cell” refers to any cell that is multipotent and derived from a multi-cellular organism after birth.

Pluripotent: As used herein, “pluripotent” refers to cells that can give rise to any cell type except the cells of the placenta or other supporting cells of the uterus.

Primordial Sex Cell: As used herein, “primordial sex cell” refers to any diploid cell that is derived from the male or female mature or developing gonad, is able to generate cells that propagate a species and contains a diploid genomic state. Primordial sex cells can be quiescent or actively dividing. These cells include male gonocytes, female gonocytes, spermatogonial stem cells, ovarian stem cells, oogonia, type-A spermatogonia, Type-B spermatogonia. Also known as germ-line stem cells.

Primordial Germ Cell: As used herein, “primordial germ cell” refers to cells present in early embryogenesis that are destined to become germ cells.

Reprogamming: As used herein “reprogramming” refers to the resetting of the genetic program of a cell such that the cell exhibits pluripotency and has the potential to produce a fully developed organism.

Responsive: As used herein, “responsive” refers to the condition of a cell, or group of cells, wherein they are susceptible to and can function accordingly within a cellular environment. Responsive cells are capable of responding to and functioning in a particular cellular environment, tissue, organ and/or organ system.

Somatic Stem Cells: As used herein, “somatic stem cells” refers to diploid multipotent or pluripotent stem cells. Somatic stem cells are not totipotent stem cells. Stem cells are primitive cells that give rise to other types of cells. Also called progenitor cells, there are several kinds of stem cells. Totipotent cells are considered the “master” cells of the body because they contain all the genetic information needed to create all the cells of the body plus the placenta, which nourishes the human embryo. Human cells have this totipotent capacity only during the first few divisions of a fertilized egg. After three to four divisions of totipotent cells, there follows a series of stages in which the cells become increasingly specialized. The next stage of division results in pluripotent cells, which are highly versatile and can give rise to any cell type except the cells of the placenta or other supporting tissues of the uterus. At the next stage, cells become multipotent, meaning they can give rise to several other cell types, but those types are limited in number. An example of multipotent cells is hematopoietic cells-blood cells that can develop into several types of blood cells, but cannot develop into brain cells. At the end of the long chain of cell divisions that make up the embryo are “terminally differentiated” cells-cells that are considered to be permanently committed to a specific function.

Therapeutic Cloning: As used herein, “therapeutic cloning” refers to the cloning of cells using nuclear transfer methods including replacing the nucleus of an ovum with the nucleus of another cell and stem cells derived from the inner cell mass.

Therapeutic Reprogramming: As used herein, “therapeutic reprogramming” refers to the process of maturation wherein a stem cell is exposed to stimulatory factors according to the teachings of the present invention to yield either pluripotent, multipotent or tissue-specific committed cells. Therapeutically reprogrammed cells are useful for implantation into a host to replace or repair diseased, damaged, defective or genetically impaired tissue. The therapeutically reprogrammed cells of the present invention do not possess non-human sialic acid residues.

In one specific embodiment, a tumor vaccine is prepared by extraction of tumor derived exosomes as a source of autologous tumor antigens, in an embodiment expressing survivin. Specifically, survivin expressing exosomes are purified from circulation using means known in the art for purification of exosomes. In one particular embodiment, exosomes are isolated from circulation using size exclusion chromatography, such as gel permeation columns, centrifugation or density gradient centrifugation, and filtration methods. For example, exosomes can be isolated by differential centrifugation, anion exchange and/or gel permeation chromatography (for example, as described in U.S. Pat. Nos. 6,899,863 and 6,812,023), sucrose density gradients, organelle electrophoresis (for example, as described in U.S. Pat. No. 7,198,923), magnetic activated cell sorting (MACS), or with a nanomembrane ultrafiltration concentrator. Various combinations of isolation or concentration methods can be used. It is known that highly abundant proteins, such as albumin and immunoglobulin, may hinder isolation of exosomes from a biological sample. For example, exosomes may be isolated from a biological sample using a system that utilizes multiple antibodies that are specific to the most abundant proteins found in blood. Such a system can remove up to several proteins at once, thus unveiling the lower abundance species such as cell-of-origin specific exosomes. This type of system can be used for isolation of exosomes from biological samples such as blood, cerebrospinal fluid or urine. The isolation of exosomes from a biological sample may also be enhanced by high abundant protein removal methods as described in Chromy et al. J. Proteome Res 2004; 3:1120-1127. In another embodiment, the isolation of exosomes from a biological sample may also be enhanced by removing serum proteins using glycopeptide capture as described in Zhang et al, Mol Cell Proteomics 2005; 4:144-155. In addition, exosomes from a biological sample such as urine may be isolated by differential centrifugation followed by contact with antibodies directed to cytoplasmic or anti-cytoplasmic epitopes as described in Pisitkun et al., Proc Natl Acad Sci USA, 2004; 101:13368-13373. Isolation or enrichment of exosomes from biological samples can also be enhanced by use of sonication (for example, by applying ultrasound), or the use of detergents, other membrane-active agents, or any combination thereof. For example, ultrasonic energy can be applied to a potential tumor site, and without being bound by theory, release of exosomes from the tissue can be increased, allowing an enriched population of exosomes that can be analyzed or assessed from a biological sample using one or more methods disclosed herein.

Subsequent to isolation of exosomes from cancer patients, a step of selectivity may be performed to enhance the number of tumor exosomes as compared to healthy exosomes. In some situations it will not be necessary to isolate out healthy exosomes because of the substantially larger number of tumor exosomes in circulation as compared to healthy exosomes.

In one embodiment a peripheral blood is extracted from a cancer patient and peripheral blood mononuclear cells (PBMC) are isolated using the Ficoll Method. PBMC are subsequently resuspended in 10 ml STEM-34 media and allowed to adhere onto a plastic surface for 2-4 hours. The adherent cells are then cultured at 37° C. in STEM-34 media supplemented with 1,000 U/mL granulocyte-monocyte colony-stimulating factor and 500 U/mL IL-4 after non-adherent cells are removed by gentle washing in Hanks Buffered Saline Solution (HBSS). Half of the volume of the GM-CSF and IL-4 supplemented media is changed every other day. Immature DCs are harvested on day 7. In one embodiment said generated DC are incubated with survivin and/or survivin peptides. These are used to stimulate T cell and NK cell tumoricidal activity. Specifically, generated DC may be further purified from culture through use of flow cytometry sorting or magnetic activated cell sorting (MACS), or may be utilized as a semi-pure population. DC may be added into said patient in need of therapy with the concept of stimulating NK and T cell activity in vivo, or in another embodiment may be incubated in vitro with a population of cells containing T cells and/or NK cells. In one embodiment DC are exposed to agents capable of stimulating maturation in vitro. Specific means of stimulating in vitro maturation include culturing DC or DC containing populations with a toll like receptor agonist. Another means of achieving DC maturation involves exposure of DC to TNF-alpha at a concentration of approximately 20 ng/mL. In order to activate T cells and/or NK cells in vitro, cells are cultured in media containing approximately 1000 IU/ml of interferon gamma. Incubation with interferon gamma may be performed for the period of 2 hours to the period of 7 days. Preferably, incubation is performed for approximately 24 hours, after which T cells and/or NK cells are stimulated via the CD3 and CD28 receptors. One means of accomplishing this is by addition of antibodies capable of activating these receptors. In one embodiment approximately, 2 ug/ml of anti-CD3 antibody is added, together with approximately 1 ug/ml anti-CD28. In order to promote survival of T cells and NK cells, was well as to stimulate proliferation, a T cell/NK mitogen may be used. In one embodiment the cytokine IL-2 is utilized. Specific concentrations of IL-2 useful for the practice of the invention are approximately 500 u/mL IL-2. Media containing IL-2 and antibodies may be changed every 48 hours for approximately 8-14 days. In one particular embodiment DC are included to said T cells and/or NK cells in order to endow cytotoxic activity towards tumor cells. In a particular embodiment, inhibitors of caspases are added in the culture so as to reduce rate of apoptosis of T cells and/or NK cells. Generated cells can be administered to a subject intradermally, intramuscularly, subcutaneously, intraperitoneally, intraarterially, intravenously (including a method performed by an indwelling catheter), intratumorally, or into an afferent lymph vessel. Together with dengue virus. Dengue virus administration is performed to enhance innate immunity and induce a cytokine storm. In a specific embodiment, autologous tumor derived exosomes are concentrated and co-administered in order to enhance antigen specific immunity. In other embodiments said exosomes are used to pulse dendritic cells in order to allow for induction of tumor immunity.

In some embodiments, the culture of the cells is performed by starting with purified lymphocyte populations, for example, The step of separating the cell population and cell sub-population containing a T cell can be performed, for example, by fractionation of a mononuclear cell fraction by density gradient centrifugation, or a separation means using the surface marker of the T cell as an index. Subsequently, isolation based on surface markers may be performed. Examples of the surface marker include CD3, CD8 and CD4, and separation methods depending on these surface markers are known in the art. For example, the step can be performed by mixing a carrier such as beads or a culturing container on which an anti-CD8 antibody has been immobilized, with a cell population containing a T cell, and recovering a CD8-positive T cell bound to the carrier. As the beads on which an anti-CD8 antibody has been immobilized, for example, CD8 MicroBeads), Dynabeads M450 CD8, and Eligix anti-CD8 mAb coated nickel particles can be suitably used. This is also the same as in implementation using CD4 as an index and, for example, CD4 MicroBeads, Dynabeads M-450 CD4 can also be used. In some embodiments of the invention, T regulatory cells are depleted before initiation of the culture. Depletion of T regulatory cells may be performed by negative selection by removing cells that express makers such as neuropilin, CD25, CD4, CTLA4, and membrane bound TGF-beta. Experimentation by one of skill in the art may be performed with different culture conditions in order to generate effector lymphocytes, or cytotoxic cells, that possess both maximal activity in terms of tumor killing, as well as migration to the site of the tumor. For example, the step of culturing the cell population and cell sub-population containing a T cell can be performed by selecting suitable known culturing conditions depending on the cell population. In addition, in the step of stimulating the cell population, known proteins and chemical ingredients, etc., may be added to the medium to perform culturing. For example, cytokines, chemokines or other ingredients may be added to the medium. Herein, the cytokine is not particularly limited as far as it can act on the T cell, and examples thereof include IL-2, IFN-gamma., transforming growth factor (TGF)-. beta., IL-15, IL-7, IFN-. alpha., IL-12, CD40L, and IL-27. From the viewpoint of enhancing cellular immunity, particularly suitably, IL-2, IFN-gamma., or IL-12 is used and, from the viewpoint of improvement in survival of a transferred T cell in vivo, IL-7, IL-15 or IL-21 is suitably used. In addition, the chemokine is not particularly limited as far as it acts on the T cell and exhibits migration activity, and examples thereof include RANTES, CCL21, MIP1.alpha., MIP1.beta., CCL19, CXCL12, IP-10 and MIG. The stimulation of the cell population can be performed by the presence of a ligand for a molecule present on the surface of the T cell, for example, CD3, CD28, or CD44 and/or an antibody to the molecule. Further, the cell population can be stimulated by contacting with other lymphocytes such as antigen presenting cells (dendritic cell) presenting a target peptide such as a peptide derived from a cancer antigen on the surface of a cell. For stimulation of antigen specific immunity, autologous exosomes from cancer patients are utilized to culture in the presence of antigen presenting cells, such as dendritic cells, and also in the presence of T cells, whose clonal expansion is desired. In addition to assessing cytotoxicity and migration as end points, it is within the scope of the current invention to optimize the cellular product based on other means of assessing T cell activity, for example, the function enhancement of the T cell in the method of the present invention can be assessed at a plurality of time points before and after each step using a cytokine assay, an antigen-specific cell assay (tetramer assay), a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. In vivo assessment of the efficacy of the generated cells using the invention may be assessed in a living body before first administration of the T cell with enhanced function of the present invention, or at various time points after initiation of treatment, using an antigen-specific cell assay, a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. Further, an immune response can be assessed by a weight, diameter or malignant degree of a tumor possessed by a living body, or the survival rate or survival term of a subject or group of subjects.

In another embodiment DC are generated from leukocytes of patients by leukopheresis. Numerous means of leukopheresis are known in the art. In one example, a Frenius Device (Fresenius Com. Tec) is utilized with the use of the MNC program, at approximately 1500 rpm, and with a P1Y kit. The plasma pump flow rates are adjusted to approximately 50 mL/min. Various anticoagulants may be used, for example ACD-A. The Inlet/ACD Ratio may be ranged from approximately 10:1 to 16:1. In one embodiment approximately 150 mL of blood is processed. The leukopheresis product is subsequently used for initiation of dendritic cell culture. In order to generated peripheral blood mononuclear cells from leukopheresis product, mononuclear cells are isolated by the Ficoll-Hypaque density gradient centrifugation. Monocytes are then enriched by the Percoll hyperosmotic density gradient centrifugation followed by two hours of adherence to the plate culture. Cells are then centrifuged at 500 g to separate the different cell populations. Adherent monocytes are cultured for 7 days in 6-well plates at 2×106 cells/mL RMPI medium with 1% penicillin/streptomycin, 2 mM L-glutamine, 10% of autologous, 50 ng/ml GM-CSF and 30 ng/mL IL-4. On approximately day 4-7 dendritic cells are admixed with patient derived exosomes in order to provide for antigen specificity. On day 7, the immature DCs are then induced to differentiate into mature DCs by culturing for 48 hours with 30 ng/mL interferon gamma (IFN-γ). During the course of generating DC for clinical purposes, microbiologic monitoring tests are performed at the beginning of the culture, on the fifth day and at the time of cell delivery.

In one embodiment of the invention pluripotent cells are transfected with additional oncogenes to survivin. Example of known oncogenes include said oncogene is selected from a group comprising of: ABCB1, ABCG2, ABI1, ABL1, ABL2, ACKR3, ACSL3, ACSL6, ACVR1B, ACVR2A, AFF1, AFF3, AFF4, AKAP9, AKT1, AKT2, AKT3, ALDH1A1, ALDH2, ALK, AMER1, ANGPT1, ANGPT2, ANKRD23, APC, AR, ARAF, AREG, ARFRP1, ARHGAP26, ARHGEF12, ARID1A, ARID1B, ARID2, ARNT, ASPSCR1, ASXL1, ATF1, ATIC, ATM, ATPIA1, ATP2B3, ATR, ATRX, AURKA, AURKB, AXIN1, AXL, BAP1, BARD1, BBC3, BCL10, BCL11A, BCL11B, BCL2, BCL2L1, BCL2L11, BCL2L2, BCL3, BCL6, BCL7A, BCL9, BCOR, BCORL1, BCR, BIRC3, BLM, BMPR1A, BRAF, BRCA1, BRCA2, BRD3, BRD4, BRINP3, BRIP1, BTG1, BTG2, BTK, BUB1B, C11orf30, C15orf65, C2orf44, CA6, CACNA1D, CALR, CAMTA1, CANT1, CARD11, CARS, CASC5, CASP8, CBFA2T3, CBFB, CBL, CBLB, CBLC, CCDC6, CCNB1IP1, CCND1, CCND2, CCND3, CCNE1, CD19, CD22, CD274, CD38, CD4, CD70, CD74, CD79A, CD79B, CD83, CDC73, CDH1, CDH11, CDK12, CDK4, CDK6, CDK7, CDK8, CDK9, CDKN1A, CDKN1B, CDKN2A, CDKN2B, CDKN2C, CDX2, CEBPA, CHCHD7, CHD2, CHD4, CHEK1, CHEK2, CHIC2, CHN1, CHORDC1, CIC, CIITA, CLP1, CLTC, CLTCL1, CNBP, CNOT3, CNTRL, COLIA1, COPB1, COX6C, CRBN, CREB1, CREB3L1, CREB3L2, CREBBP, CRKL, CRLF2, CRTC1, CRTC3, CSF1R, CSF3R, CTCF, CTLA4, CTNNA1, CTNNB1, CUL3, CXCR4, CYLD, CYP17A1, CYP2D6, DAXX, DDB2, DDIT3, DDR1, DDR2, DDX10, DDX3X, DDX5, DDX6, DEK, DICER1, DIS3, DLL4, DNM2, DNMT1, DNMT3A, DOT1L, DPYD, DUSP4, DUSP6, EBF1, ECT2L, EDNRB, EED, EGFR, EIF4A2, ELF4, ELK4, ELL, ELN, EML4, EP300, EPHA3, EPHA5, EPHA7, EPHA8, EPHB1, EPHB2, EPHB4, EPS15, ERBB2, ERBB3, ERBB4, ERC1, ERCC1, ERCC2, ERCC3, ERCC4, ERCC5, EREG, ERG, ERN1, ERRFI1, ESR1, ETV1, ETV4, ETV5, ETV6, EWSR1, EXT1, EXT2, EZH2, EZR, FAF1, FAIM3, FAM46C, FANCA, FANCC, FANCD2, FANCE, FANCF, FANCG, FANCL, FAS, FAT1, FBXO11, FBXW7, FCRL4, FEV, FGF10, FGF14, FGF19, FGF2, FGF23, FGF3, FGF4, FGF6, FGFR1, FGFR1OP, FGFR2, FGFR3, FGFR4, FH, FHIT, FIP1L1, FKBP1A, FLCN, FLI1, FLT1, FLT3, FLT4, FNBP1, FOXA1, FOXL2, FOXO1, FOXO3, FOXO4, FOXP1, FRS2, FSTL3, FUBP1, FUS, GABRA6, GAS7, GATA1, GATA2, GATA3, GATA4, GATA6, GID4, GLI1, GMPS, GNA11, GNA12, GNA13, GNAQ, GNAS, GNRH1, GOLGA5, GOPC, GPC3, GPHN, GPR124, GRIN2A, GRM3, GSK3B, GUCY2C, H3F3A, H3F3B, HCK, HDAC1, HERPUD1, HEY1, HGF, HIP1, HIST1H1E, HIST1H3B, HIST1H4I, HLF, HMGA1, HMGA2, HMGN2P46, HNF1A, HNMT, HNRNPA2B1, HNRNPK, HOOK3, HOXA11, HOXA13, HOXA9, HOXC11, HOXC13, HOXD11, HOXD13, HRAS, HSD3B1, HSP90AA1, HSP90AB1, IAPP, ID3, IDH1, IDH2, IGF1R, IGF2, IKBKE, IKZF1, IL2, IL21R, IL3RA, IL6, IL6ST, IL7R, INHBA, INPP4B, IRF2, IRF4, IRS2, ITGAV, ITGB1, ITK, ITPKB, JAK1, JAK2, JAK3, JAZF1, JUN, KAT6A, KAT6B, KCNJ5, KDM1A, KDM5A, KDM5C, KDM6A, KDR, KDSR, KEAP1, KEL, KIAA1549, KIF5B, KIR3DL1, KIT, KLF4, KLHL6, KLK2, KMT2A, KMT2C, KMT2D, KRAS, KTN1, LASP1, LCK, LCP1, LGALS3, LGR5, LHFP, LIFR, LMO1, LMO2, LOXL2, LPP, LRIG3, LRP1B, LUC7L2, LYL1, LYN, LZTR1, MAF, MAFB, MAGED1, MAGI2, MALT1, MAML2, MAP2K1, MAP2K2, MAP2K4, MAP3K1, MAPK1, MAPK11, MAX, MCL1, MDM2, MDM4, MDS2, MECOM, MED12, MEF2B, MEN1, MET, MITF, MKI67, MKL1, MLF1, MLH1, MLLT1, MLLT10, MLLT11, MLLT3, MLLT4, MLLT6, MMP9, MN1, MNX1, MPL, MRE11A, MS4A1, MSH2, MSH6, MSI2, MSN, MST1R, MTCP1, MTF2, MTOR, MUC1, MUC16, MUTYH, MYB, MYC, MYCL, MYCN, MYD88, MYH11, MYH9, NACA, NAE1, NBN, NCKIPSD, NCOA1, NCOA2, NCOA4, NDRG1, NF1, NF2, NFE2L2, NFIB, NFKB2, NFKBIA, NIN, NKX2-1, NONO, NOTCH1, NOTCH2, NOTCH3, NPM1, NR4A3, NRAS, NSD1, NT5C2, NTRK1, NTRK2, NTRK3, NUMA1, NUP214, NUP93, NUP98, NUTM1, NUTM2B, OLIG2, OMD, P2RY8, PAFAH1B2, PAK3, PALB2, PARK2, PARP1, PATZ1, PAX3, PAX5, PAX7, PAX8, PBRM1, PBX1, PCM1, PCSK7, PDCD1, PDCD1LG2, PDE4DIP, PDGFB, PDGFRA, PDGFRB, PDK1, PECAM1, PER1, PHF6, PHOX2B, PICALM, PIK3C2B, PIK3CA, PIK3CB, PIK3CD, PIK3CG, PIK3R1, PIK3R2, PIM1, PLAG1, PLCG2, PML, PMS1, PMS2, POLD1, POLE, POT1, POU2AF1, POU5F1, PPARG, PPP2R1A, PRCC, PRDM1, PRDM16, PREX2, PRF1, PRKAR1A, PRKCI, PRKDC, PRLR, PRPF40B, PRRT2, PRRX1, PRSS8, PSIP1, PSMD4, PTBP1, PTCH1, PTEN, PTK2, PTPN11, PTPRC, PTPRD, QK1, RABEP1, RAC1, RAD21, RAD50, RAD51, RAD51B, RAD51C, RAD51D, RAF1, RALGDS, RANBP17, RANBP2, RAP1GDS1, RARA, R131, RBM10, RBM15, RCOR1, RECQL4, REL, RELN, RET, RHOA, RHOH, RICTOR, RIPK1, RM12, RNF213, RNF43, ROS1, RPL10, RPL22, RPL5, RPN1, RPS6KB1, RPTOR, RUNX1, RUNX1T1, S1PR2, SAMHD1, SBDS, SDC4, SDHA, SDHAF2, SDHB, SDHC, SDHD, SEPT5, SEPT6, SEPT9, SET, SETBP1, SETD2, SF1, SF3A1, SF3B1, SF3B2, SFPQ, SGK1, SH2B3, SH3GL1, SLAMF7, SLC34A2, SLC45A3, SLIT2, SMAD2, SMAD3, SMAD4, SMARCA4, SMARCB1, SMARCE1, SMC1A, SMC3, SMO, SNCAIP, SNX29, SOCS1, SOX10, SOX11, SOX2, SOX9, SPECC1, SPEN, SPOP, SPTA1, SRC, SRGAP3, SRSF2, SRSF3, SS18, SS18L1, SSX1, STAG2, STAT3, STAT4, STATSB, STEAP1, STIL, STK11, SUFU, SUZ12, SYK, TAF1, TAF15, TAL1, TAL2, TBL1XR1, TBX3, TCEA1, TCF12, TCF3, TCF7L2, TCL1A, TEK, TERC, TERT, TET1, TET2, TFE3, TFEB, TFG, TFPT, TFRC, TGFB1, TGFBR2, THRAP3, TIMP1, TJP1, TLX1, TLX3, TM7SF2, TMPRSS2, TNFAIP3, TNFRSF14, TNFRSF17, TNFRSF18, TNFRSF9, TNFSF11, TOP1, TOP2A, TP53, TP63, TPBG, TPM3, TPM4, TPR, TRAF2, TRAF3, TRAF3IP3, TRAF7, TRIM26, TRIM27, TRIM33, TRIP11, TRRAP, TSC1, TSC2, TSHR, TTK, TTL, TYMS, U2AF1, U2AF2, UBA1, UBR5, USP6, VEGFA, VEGFB, VHL, VPS51, VTI1A, WAS, WEE1, WHSC1, WHSC1L1, WIF1, WISP3, WNT11, WNT2B, WNT3, WNT3A, WNT4, WNT5A, WNT6, WNT7B, WRN, WT1, WWTR1, XBP1, XPA, XPC, XPO1, YWHAE, YWHAZ, ZAK, ZBTB16, ZBTB2, ZMYM2, ZMYM3, ZNF217, ZNF331, ZNF384, ZNF521, ZNF703 and ZRSR2

### Working Example: Generation of Immune Stimulator Dendritic Cell Derived Exosomes from TGF-Beta Silenced Progenitor Cells

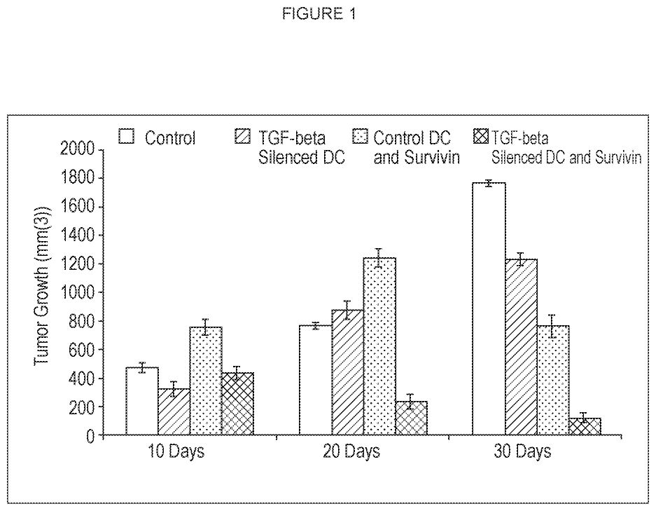

Induced pluripotent stem cells (C57BL/6 backround SCRC 1002) cells were resuspended in RPMI 1640 in 10% fetal calf serum with pen/strept mixture (Thermo Fisher) at a concentration of 1 million cells per ml and plated in 6 well plates. Cells were cultured in M-CSF to induce monocytic differentiation. Adherent monocyte cells were subsequently collected by trypsinization and transfected with siRNA to TGF-beta (sense, GCAACAACGCCAUCUAUGA (SEQ ID NO: 1); antisense, UCAUAGAUGGCGUUGUUGC (SEQ ID NO: 2)) from Sigma-Aldrich (St. Louis, MO). Transfection was performed using lipofectamine based methodology as published by Hill et al J Immunol 171:691, 2003. Monocytes were converted to dendritic cells by culture in 100 IU/ml IL-4 and 100 IU/ml GM-CSF and cultured for a total of 7 days. On day 5 of culture cells where pulsed with survivin peptide (QIWQLYLKNYRIATFKNWP (SEQ ID NO: 3) or scrambled peptide (YLKNYRIQIWQLATFKNWP (SEQ ID NO: 6) (100 ng/ml). On day 7 conditioned media was extracted and exosomes were concentrated using the Exoquick kit from control DC, siRNA TGF-beta treated DC, control DC with survivin peptide, and siRNA TGF-beta DC with survivin peptide. Exosomes (1 ug/mouse) were administered to C57/BL6 mice (10 per group) bearing B16 melanoma at week 1 after administration of 500,000 B16 cells intradermally daily for 3 days. Results are shown in FIG. 1.

1. A composition useful for stimulating an immune response to cancer comprising exosomes produced by dendritic cells expressing survivin or parts of the survivin gene, wherein said dendritic cells are derived from a cell type that was dedifferentiated into immature cells which are subsequently used to generate a dendritic cell population, wherein during said immature phase various immunomodulatory modifications have been made to said immature cells, furthermore wherein said modified immature cells are expanded to generate a master cell bank and subsequently said immature cells are differentiated to dendritic cells and said dendritic cells are utilized as a source of exosomes.

2. The composition of claim 1, wherein said dendritic cells are generated from pluripotent stem cells.

3. The composition of claim 2, wherein said pluripotent stem cells are induced pluripotent stem cells.

4. The composition of claim 1, wherein said pluripotent stem cells are differentiated into monocytes.

5. The composition of claim 4, wherein said monocytes are differentiated into dendritic cells.

6. The composition of claim 4, wherein said pluripotent stem cells are differentiated into monocytes by culture in GM-CSF and IL-4.

7. The composition of claim 5, wherein said dendritic cells express CD11c.

8. The composition of claim 5, wherein said dendritic cells express CD40.

9. The composition of claim 5, wherein said dendritic cells express CD80.

10. The composition of claim 5, wherein said dendritic cells express CD86.

11. The composition of claim 5, wherein said dendritic cells express HLAII.

12. The composition of claim 5, wherein said dendritic cells express DEC-205.

13. The composition of claim 4, wherein said monocytes are silenced for expression of TGF-beta.

14. The composition of claim 13, wherein said monocytes are silenced for expression of TGF-beta by administration of siRNA sequence comprising of (sense, GCAACAACGCCAUCUAUGA (SEQ ID NO: 1); antisense, UCAUAGAUGGCGUUGUUGC (SEQ ID NO: 2).

15. The composition of claim 12, wherein said dendritic cells are pulsed with a survivin peptide.

16. The composition of claim 15, wherein said survivin peptide is QIWQLYLKNYRIATFKNWP (SEQ ID NO: 3).

17. The composition of claim 15, wherein said survivin peptide is ATFKNWPF (SEQ ID NO: 4).

18. The composition of claim 15, wherein said survivin peptide is AKFVAAWTLKAAA (SEQ ID NO: 5).

19. The composition of claim 15, wherein pulsed dendritic cells are used as a source of exosomes.

20. The composition of claim 19, wherein said exosomes are administered as a cancer therapeutic.


SEQUENCE LISTING (6 sequences)

SEQ ID NO: 1 - RNA, 19 nt, synthetic construct
  source 1..19: mol_type = other RNA
gcaacaacgccatctatga

SEQ ID NO: 2 - RNA, 19 nt, synthetic construct
  source 1..19: mol_type = other RNA
tcatagatggcgttgttgc

SEQ ID NO: 3 - protein, 19 aa, synthetic construct
QIWQLYLKNYRIATFKNWP

SEQ ID NO: 4 - protein, 8 aa, synthetic construct
ATFKNWPF

SEQ ID NO: 5 - protein, 13 aa, synthetic construct
AKFVAAWTLKAAA

SEQ ID NO: 6 - protein, 19 aa, synthetic construct
YLKNYRIQIWQLATFKNWP